(19) United States
(12) Patent Application Publication
(10) Pub. No.: US 2024/0269208 A1
(43) Pub. Date: Aug. 15, 2024

(54) METHOD FOR PRODUCING PLANT-DERIVED COLLAGEN PEPTIDE MIXTURE

(71) Applicant: RAWGA Co., Ltd., Seongnam-si (KR)

(72) Inventors: Tae Un Ha, Gapyeong-gun (KR); Hyun Min Kim, Seongnam-si (KR); Boo Sik Jang, Cheongju-si (KR)

(21) Appl. No.: 18/507,651

(22) Filed: Nov. 13, 2023

(30) Foreign Application Priority Data
Feb. 10, 2023 (KR) 10-2023-0018123

(51) Int. Cl. A61K 36/185; A61K 36/07; A61K 36/28; A61K 36/31; A61K 36/488

(52) U.S. Cl. CPC A61K 36/185; A61K 36/07; A61K 36/28; A61K 36/31; A61K 36/488; A61K 2236/19; A61K 2236/331

(57) ABSTRACT

The present disclosure relates to a method for producing a plant-derived collagen peptide mixture, the method including steps of: mixing Hibiscus and Tremella fuciformis; adding water to the mixture, followed by heat treatment at 70 to 90° C. for 30 to 60 minutes; adding an acidic solution to the heat-treated product, and adding protease thereto, followed by hydrolysis at 40 to 60° C. for 2 to 24 hours; and deactivating the protease by heating the hydrolyzed solution at 75 to 85° C. The plant-derived collagen peptide mixture produced according to the method of the present disclosure has excellent safety and reliability, is easy to take because it has no fishy smell, has a high absorption rate in vivo, and promotes collagen synthesis in vivo, thereby exhibiting excellent anti-aging and anti-wrinkle effects and fatigue recovery effects.

### BACKGROUND

### 1. Technical Field

The present disclosure relates to a method for producing a plant-derived collagen peptide mixture, and more particularly, to a method of producing a plant-derived collagen peptide mixture by extracting a collagen peptide mixture from Hibiscus and Tremella fuciformis, wherein the plant-derived collagen peptide mixture has excellent safety and reliability, and a high absorption rate in the human body, and exhibits excellent effects such as anti-aging and anti-wrinkle effects and fatigue recovery effects when taken.

### 2. Related Art

Collagen is the fibrous protein most abundant in the animal body, and is the main component of the dermal layer and connective tissue of the skin, and 90% of the proteins that make up bone are collagen. In general, collagen, which is used for medical, cosmetic and food applications, is extracted from animal bones and skin, and is a polymer protein composed of three polypeptide chains with a molecular weight of about 100,000, jointed together in a helical structure.

Collagen is a polypeptide that has a molecular weight of about 130,000 and accounts 1/3 of mammalian tissue. It is the primary constituent in the skin and the connective tissue, and is part of organic substances in bones and teeth. It is produced by dissolving the mineral parts of bone with phosphoric acid.

A method of isolating collagen from tissue includes organic solvent extraction, treatment with acid and alkali, and then treatment with trypsin and hyaluronidase to obtain collagen, which is an insoluble substance. Amino acids that constitute collagen include proline, oxyproline (hydroxyproline), glycine, glutamic acid, and the like.

Studies on anti-aging have recently been actively conducted due to a rapid increase in the elderly population, and the desire for youth, regardless of age or gender, is difficult to control. With this boom of the times, the number of people looking for collagen has increased as the efficacy and effects of collagen have been reported.

In particular, in the functional food field, a number of functional food compositions for anti-aging containing collagen have been released.

However, most of the collagen currently in distribution is animal or marine collagen. As diseases such as foot-and-mouth disease and mad cow disease spread and marine pollution intensifies, anxiety about animal or marine collagen has intensified, and thus the reliability of animal or marine collagen is not high. In addition, the animal or marine collagen has a problem in that it is difficult to take due to its unique fishy smell.

Accordingly, there is a need to overcome the vulnerability of animal or marine collagen, which has been used with anxiety, by developing a plant-derived protein (hereinafter referred to as “plant-derived collagen”) containing hydroxyproline, proline, lysine, and the like, which are the main components of collagen.

Prior art documents relating to food compositions containing such plant-derived collagen include Korean Patent No. 10-0827389 and Korean Patent No. 10-0488913. The above patents propose a method of producing collagen peptides from plant-derived or marine raw materials. In addition, the present applicant proposed a food composition containing collagen peptides extracted from Hibiscus (Korean Patent No. 10-2328107).

However, no method of producing a collagen peptide mixture by extraction from Hibiscus and Tremella fuciformis could be found in any of the above-described patents.

### Prior Art Documents

### [Patent Documents]

(Patent Document 0001) KR 10-0827389 B1
(Patent Document 0002) KR 10-0488913 B1
(Patent Document 0003) KR 10-2328107 B1

### SUMMARY

Therefore, an object of the present disclosure is to provide a method of producing a plant-derived collagen peptide mixture by extracting a plant-derived collagen peptide mixture from Hibiscus and Tremella fuciformis, wherein the plant-derived collagen peptide mixture is easy to take because it has no fishy smell, and promotes collagen synthesis in vivo, thereby exhibiting skin anti-aging and anti-wrinkle effects, skin whitening effects, and fatigue recovery effects.

Another object of the present disclosure is to provide a method of producing a plant-derived collagen peptide mixture, which has excellent effects of promoting collagen synthesis in vivo and reducing fatigue, by combining an extracted plant-derived collagen peptide mixture with a natural product fermentation extract and a natural product extract.

A method for producing a plant-derived collagen peptide mixture according to the present invention includes steps of: mixing Hibiscus and Tremella fuciformis; adding water to the mixture, followed by heat treatment at 70 to 90° C. for 30 to 60 minutes; adding an acidic solution to the heat-treated product, and adding protease thereto, followed by hydrolysis at 40 to 60° C. for 2 to 24 hours; and deactivating the protease by heating the hydrolyzed solution at 75 to 85° C.

The step of mixing Hibiscus and Tremella fuciformis may include mixing Hibiscus and Tremella fuciformis at a weight ratio of 1:0.5 to 1.

The method may further include, after the step of deactivating the protease, a step of mixing a fermentation extract of Pueraria flos and an extract of chicory with the deactivated solution.

The step of mixing the fermentation extract of Pueraria flos and the extract of chicory with the deactivated solution may further include mixing an extract of kohlrabi.

The plant-derived collagen peptide mixture may promote collagen synthesis in vivo.

The plant-derived collagen peptide mixture produced according to the method of the present disclosure has excellent safety and reliability, is easy to take because it has no fishy smell, has a high absorption rate in vivo, and promotes collagen synthesis in vivo, thereby exhibiting excellent anti-aging and anti-wrinkle effects and fatigue recovery effects.

### BRIEF DESCRIPTION OF THE DRAWINGS

FIG. 1 is a flow chart showing a method for producing a plant-derived collagen peptide mixture according to the present disclosure.

FIG. 2 is a graph showing the results of Test Example 5 according to the present disclosure.

### DETAILED DESCRIPTION

Hereinafter, the present disclosure will be described in detail.

The present disclosure relates to a method for producing a plant-derived collagen peptide mixture which promotes collagen synthesis in vivo, thereby preventing and ameliorating aging or reducing fatigue.

A method for producing a plant-derived collagen peptide mixture according to the present invention includes steps of: mixing Hibiscus and Tremella fuciformis; adding water to the mixture, followed by heat treatment at 70 to 90° C. for 30 to 60 minutes; adding an acidic solution to the heat-treated product, and adding protease thereto, followed by hydrolysis at 40 to 60° C. for 2 to 24 hours; and deactivating the protease by heating the hydrolyzed solution at 75 to 85° C.

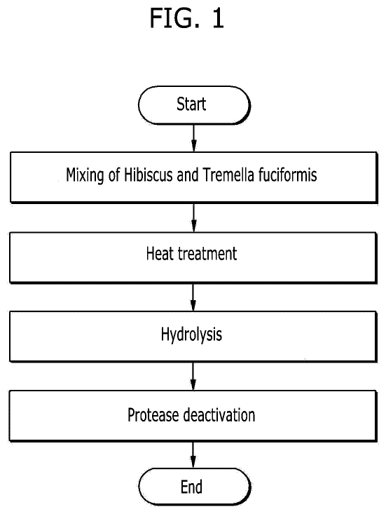

Hereinafter, each step of the method according to the present disclosure will be described in detail with reference to FIG. 1.

Step of Mixing Hibiscus and Tremella fuciformis

First, as a raw material, Hibiscus fruits, flowers, or mixtures thereof are washed and prepared. Tremella fuciformis is also washed and prepared. Here, it is to be understood that dried products of Hibiscus and Tremella fuciformis may also be used.

Hibiscus is a general name for dicotyledonous plants belonging to the order Malvales, the family Malvaceae and the genus Hibiscus, and is known to contain biologically active substances useful for the human body, which promote the rapid excretion of waste products when taken and are effective against arteriosclerosis, cancer, enteritis, bronchitis, etc. In addition, it is known that pyruvic acid in a Hibiscus sabdariffa extract, when taken together with DHA (dihydroxyacetone), has not only a weight loss effect by promotion of lipolysis, but also a protein-degrading effect. In addition, it is known that Hibiscus, when applied as a patch to the skin, degrades proteins, thereby removing dead skin cells and increasing skin regeneration rate, and blocks and suppresses reactive oxygen species, thereby preventing skin aging, and vitamin C abundantly contained therein alleviates skin troubles such as freckles, thereby exhibiting an excellent skin care effect.

Tremella fuciformis belongs to the order Tremellales, and the fruit body (mushroom) thereof is agar-like and is known to have various effects such as enhancing immunity, improving bone health, alleviating constipation, anticancer, skin care, preventing vascular disease, and preventing anemia.

In addition, Hibiscus and Tremella fuciformis are mixed together at a ratio of 1:0.5 to 1.5. In the present disclosure, the reason why Hibiscus and Tremella fuciformis are used in a mixed state rather than used alone is because when they are used alone, the effect of promoting collagen synthesis is not high and the extraction yield is also not good.

### Step of Adding Water to the Mixture, Followed by Heat Treatment at 70 to 90° C. for 30 to 60 Minutes

Then, a 3- to 5-fold weight of water is added to the mixture and chopping the mixture with a chopper. Here, the size of the chopped mixture may be about 10 to 200 μm, without being limited thereto.

Next, the mixture is heat-treated at 70 to 100° C. for 30 to 60 minutes.

Step of Adding an Acidic Solution to the Heat-Treated Product, and Adding Protease Thereto, Followed by hydrolysis at 40 to 60° C. for 2 to 24 hours

Then, at least one organic acid selected from among acetic acid, malic acid, maleic acid, succinic acid, and citric acid is added to the heat-treated product to adjust the pH to 3.0 to 6.0. Since the heat-treated product can be easily hydrolyzed even in a weakly acidic state rather than a strongly acidic state, the pH is adjusted to about 3.0 to 6.0.

Then, at least one protease selected from the group consisting of collagenase, trypsin, papain, pepsin, and alcalase is added to the pH-adjusted product, followed by hydrolysis at 40 to 60° C. for 2 to 24 hours. This is because when the hydrolysis reaction occurs under temperature and time conditions within the above ranges, it is possible to obtain a collagen peptide mixture with an appropriate molecular weight and size and a high absorption rate in vivo.

In this case, the amount of protease added may be an amount known in the art to which the present disclosure pertains, without being limited thereto.

### Step of Deactivating the Protease by Heating the Hydrolyzed Solution at 75 to 85° C.

Next, the protease is deactivated by heating the hydrolyzed solution at 75 to 85° C. for 10 to 60 minutes. Then, the resulting solution is filtered. Here, the filtration method is not limited.

Then, the filtrate is concentrated or dried as needed.

The plant-derived collagen peptide mixture produced as described above has a weight-average molecular weight of 300 to 500 daltons (Da), has a significantly high absorption rate in vivo, and is rich in components such as proline, hydroxyproline, and lysine, which effectively promote collagen synthesis in vivo and exhibit excellent anti-aging, anti-wrinkle, skin whitening, and fatigue recovery effects.

In addition, this plant-derived collagen peptide mixture promotes collagen synthesis in fibroblasts, thereby exhibiting effects such as increasing skin elasticity, reducing wrinkles and suppressing skin aging.

Meanwhile, the method preferably further includes, after the step of deactivating the protease, a step of mixing a fermentation extract of Pueraria flos and an extract of chicory with the deactivated solution.

When the fermentation extract of Pueraria flos and the extract of chicory are mixed with the deactivated solution, there is an advantage in that they further promote collagen synthesis in fibroblasts, thereby doubling the effect, and the effect of relieving fatigue is more remarkable.

Pueraria flos is a light purple flower of the perennial vine plant Pueraria thunbergina, and a fermentation extract thereof is prepared by fermenting a hot water extract of Pueraria flos with Aspergillus oryzae at 37 to 45° C. for 7 to 10 hours, then drying the same at 50 to 80° C. for 15 to 24 hours, followed by pulverization.

As chicory, the root or whole plant thereof is used. The extract of chicory is preferably obtained by extraction using at least one of water and an alcohol with 1 to 4 carbon atoms as an extraction solvent, and the extraction may be performed using any conventional method known in the art, without being limited thereto.

In addition, the deactivated solution, the fermentation product of Pueraria flos, and the extract of chicory may be included at a weight ratio of 1:0.1 to 0.5:0.1 to 0.5 (on a solids basis), and in this case, they exhibit excellent effects of promoting collagen synthesis in fibroblasts and reducing fatigue.

In addition, the step of mixing the fermentation extract of Pueraria flos and the extract of chicory with the deactivated solution preferably further includes mixing an extract of kohlrabi. In this case, the effect of reducing fatigue is increased by the extract of kohlrabi.

The extract of kohlrabi is preferably obtained by extracting the bulb of kohlrabi using at least one of water and an alcohol with 1 to 4 carbon atoms as an extraction solvent, and the extraction may be performed using any conventional method known in the art, without being limited thereto.

In this case, the extract of kohlrabi may be added in such an amount that the deactivated solution, the fermentation product of Pueraria flos, the extract of chicory, and the extract of kohlrabi are at a weight ratio of 1:0.1 to 0.5:0.1 to 0.5:0.1 to 0.5 (on a solids basis).

Meanwhile, the plant-based collagen peptide mixture produced according to the present disclosure may be prepared into a food composition.

The food composition is not particularly limited and includes a health functional food composition. When the health functional food composition of the present disclosure is used as a food additive, the composition may be added alone or used together with other foods or food ingredients, and may be used appropriately according to conventional methods. The food is not limited to a particular type and includes any food in the usual sense. Non-limiting examples of foods to which the plant-derived collagen peptide mixture may be added include meat, sausages, bread, chocolate, candies, snacks, confectionery, pizza, ramen, other noodles, gum, dairy products including ice cream, various soups, and beverages, tea, drinks, alcoholic beverages, and vitamin complexes.

When the health functional food composition is a beverage composition, it may further contain various flavoring agents or natural carbohydrates, like common beverages. Non-limiting examples of the natural carbohydrates include monosaccharides such as glucose and fructose; disaccharides such as maltose and sucrose; natural sweeteners such as dextrin and cyclodextrin; and synthetic sweeteners such as saccharin and aspartame. The proportion of the additional components to be added may be appropriately selected and determined by those skilled in the art.

In addition to the above components, the health functional food composition of the present disclosure may contain various nutrients, vitamins, electrolytes, flavoring agents, colorants, pectic acid and its salts, alginic acid and its salts, organic acids, protective colloidal thickeners, pH adjusting agents, stabilizers, preservatives, glycerin, alcohol, carbonating agents that are used in carbonated drinks, etc. In addition, the health functional food composition of the present disclosure may contain fruit flesh that is used for the production of natural fruit juice, fruit juice beverages or vegetable beverages. These components may be used independently or in combination of two or more. The proportion of these additives may also be appropriately selected by those skilled in the art.

The food composition of the present disclosure may be taken in an appropriate amount selected depending on age and weight.

In addition, the plant-derived collagen peptide mixture produced according to the present disclosure may be prepared into a cosmetic composition.

In this case, the cosmetic composition is not limited to a particular type, and it is to be understood that the cosmetic composition may be prepared according to any conventional method known in the art to which the present disclosure, and may further contain various known additives.

Hereinafter, the present disclosure will be described in detail with reference to examples.

### Example 1

0.5 kg of Hibiscus fruits and 0.5 kg of a dried product of Tremella fuciformis were mixed together, and 4 L of water was added thereto. Then, the mixture was pulverized to a size of 10 to 100 μm with a grinder and heat-treated at 80° C. for 60 minutes.

Then, the heat-treated product was adjusted to a pH of 3.5 by adding malic acid thereto, and 5 g of trypsin as protease was added thereto, followed by hydrolysis at 50° C. for 10 hours. The hydrolyzed solution was heated at 80° C. for 30 minutes to deactivate the trypsin and stirred in a centrifuge at 1,000 rpm for 30 minutes, thereby producing a plant-derived collagen peptide mixture. Then, the collagen peptide mixture was freeze-dried.

### Example 2

The freeze-dried product of plant-derived collagen peptide mixture produced in Example 1, a fermentation extract of Pueraria flos and an extract of chicory were mixed together at a weight ratio of 1:0.2:0.2.

In this case, the fermentation extract of Pueraria flos was prepared by adding a 10-fold weight of water to Pueraria flos, performing extraction at 80° C. for 5 hours, filtering the extract, inoculating the filtrate with 0.5% (v/v) of Aspergillus oryzae (1×10⁷ cfu/g), and performing fermentation at 40° C. for 10 hours, followed by drying at 70° C. for 20 hours and pulverization to a size of 200 mesh.

In addition, the extract of chicory was prepared by adding a 10-fold weight of water to the whole plant of chicory, and performing extraction at 80° C. for 5 hours, followed by filtration and freeze-drying.

### Example 3

The same process as described in Example 2 was performed, except that the freeze-dried product of plant-derived collagen peptide mixture produced in Example 1, the fermentation extract of Pueraria flos, the extract of chicory, and an extract of kohlrabi were mixed together at a weight ratio of 1:0.2:0.2:0.2.

The extract of chicory was prepared by adding a 10-fold weight of water to the bulb of kohlrabi, and performing extraction at 80° C. for 5 hours, followed by filtration and freeze-drying.

### Comparative Example 1

1 kg of Hibiscus fruits were prepared and 4 L of water was added thereto. Then, the mixture was pulverized to a size of 10 to 100 μm with a grinder and heat-treated at 80° C. for 60 minutes.

Then, the heat-treated product was adjusted to a pH of 3.5 by adding malic acid thereto, and 5 g of trypsin as protease was added thereto, followed by hydrolysis at 50° C. for 10 hours. The hydrolyzed solution was heated at 80° C. for 30 minutes to deactivate the trypsin and stirred in a centrifuge at 1,000 rpm for 30 minutes, thereby producing a Hibiscus plant-derived collagen peptide mixture. Then, the collagen peptide mixture was freeze-dried.

### Test Example 1

Total amino acid analysis of the plant-derived collagen peptide mixture produced in Example 1 was performed. The total amino acid analysis was performed using the total amino acid composition & OH-Proline analysis method. In addition, monosaccharide analysis was performed. The results are shown in Tables 1 and 2 below.

TABLE 1
Results of amino acid analysis in Test Example 1

| | Content | | Content |
|---|---|---|---|
| Amino acid | (%) | Amino acid | (%) |
| Aspartic acid (Asp) | 31.95 | Proline (Pro) | 4.45 |
| Glutamic acid (Glu) | 10.04 | Tyrosine (Tyr) | 1.14 |
| Hydroxyproline (Hyp) | 1.13 | Valine (Val) | 4.63 |
| Serine (Ser) | 6.90 | Methionine (Met) | 1.31 |
| Glycine (Gly) | 11.20 | Isoleucine (Ile) | 6.01 |
| Histidine (His) | 1.11 | Leucine (Leu) | 3.34 |
| Arginine (Arg) | 2.38 | Phenylalanine (Phe) | 2.05 |
| Threonine (Thr) | 3.42 | Lysine (Lys) | 2.15 |
| Alanine (Ala) | 6.79 | | |

TABLE 2
Results of monosaccharide composition
analysis in Test Example 1

| Sugar | Content (%) |
|---|---|
| Glucose | 3.51 |
| Mannose | 1.01 |
| Galactose | 1.54 |
| Fucose | 0.06 |
| Galactosamine | 0.02 |
| Glucosamine | 0.07 |
| N-acetylneuraminic | 0.05 |
| acid (sialic acid) | |
| N-glycolylneuraminic | 0.02 |
| acid (sialic acid) | |

As shown in Tables 1 and 2 above, it was confirmed that the sample of Example 1 was rich in hydroxyproline, lysine, proline, etc., and was rich in glutamic acid, aspartic acid, arginine, etc., which are effective in preventing skin aging, and contained small amounts of monosaccharides.

### Test Example 2

The molecular weight of the plant-derived collagen peptide mixture produced in Example 1 was measured.

As a result, it was confirmed that the plant-derived collagen peptide mixture had a molecular weight of 420 Da, indicating that the absorption rate thereof would be excellent.

### Test Example 3

In order to evaluate the skin whitening effect of each sample, B16-F10 melanocytes were treated with various concentrations of each sample, and then the tyrosinase inhibitory activity of the sample was measured.

To prepare melanocytes for testing, 500 μL of a supernatant of B16-F10 melanocytes (ATCC lot number 60508145) (50,000 cells/well) was seeded into each well of a 24-well plate and pre-incubated at 37° C. under 5% CO₂ for 24 hours so as to adhere to the well plate.

Next, samples were prepared by diluting each of the collagen peptide mixtures of Examples 1, 2 and 3 and Comparative Example 1, and vitamin C as a positive control in water at concentrations of 0.2 mg/ml, 1.0 mg/ml, and 5.0 mg/ml, and 50 μL of each sample was dispensed into each well and incubated at 37° C. under 5% CO₂ for 24 hours. Next, each well was treated with 10 μL of 1 mg/ml trypsin and incubated for 10 minutes, followed by centrifugation at 1,000 rpm for about 2 minutes to obtain a pellet. The obtained pellet was dissolved in 0.5 ml of 1% triton X100 in PBS, and 0.5 ml of 0.2% L-DOPA in 0.1M sodium phosphate buffer was added thereto, followed by incubation at 37° C. for 2 hours, and the absorbance at 490 nm was measured. Tyrosinase activity inhibition rate (%) was calculated using the following equation, and the results of measuring the tyrosinase activity inhibition rate (%) are shown in Table 3 below.

$$Tyrosinase activity inhibition rate ( \% ) = \{ ( absorbance of control group - absorbance of sample treated group ) \text{} / absorbance of control group \} \times 100$$

TABLE 3
Results of Test Example 3

| | | Tyrosinase activity |
|---|---|---|
| | | inhibition rate (%) |
| Example 1 | 0.2 mg/ml | 21.51 |
| | 1.0 mg/ml | 35.17 |
| | 5.0 mg/ml | 39.14 |
| Example 2 | 0.2 mg/ml | 22.43 |
| | 1.0 mg/ml | 36.98 |
| | 5.0 mg/ml | 40.67 |
| Example 3 | 0.2 mg/ml | 22.52 |
| | 1.0 mg/ml | 37.01 |
| | 5.0 mg/ml | 41.29 |
| Comparative | 0.2 mg/ml | 18.25 |
| Example 1 | 1.0 mg/ml | 30.15 |
| | 5.0 mg/ml | 35.21 |
| Vitamin C | 0.2 mg/ml | 27.12 |
| | 1.0 mg/ml | 56.25 |
| | 5.0 mg/ml | 70.25 |

As shown in Table 3 above, it could be confirmed that the samples of Examples 1, 2 and 3 had excellent tyrosinase activity inhibition rates, and suggesting that they have a skin whitening effect.

### Test Example 4

In order to evaluate the skin anti-wrinkle effect of each sample, B16-F10 melanocytes were treated with each sample at various concentrations, and then the elastase inhibitory activity of each sample was measured.

To prepare melanocytes for testing, 500 μL of a supernatant of B16-F10 melanocytes (ATCC lot number 60508145) (50,000 cells/well) was seeded into each well of a 24-well plate and pre-incubated at 37° C. under 5% CO₂ for 24 hours so as to adhere to the well plate.

Next, samples were prepared by diluting each of the collagen peptide mixtures of Examples 1, 2 and 3 and Comparative Example 1, and vitamin C as a positive control in water at concentrations of 0.2 mg/ml, 1.0 mg/ml, and 5.0 mg/ml, and 10 μL of each sample was dispensed into each well. Then, 50 μL of porcine pancreas elastase (10 μg/mL) in 50 mM Tris-HCl buffer (pH 8.6) was added to each well, and 100 μL of N-succinyl-(L-Ala)3-p-nitroanilide (0.5 mg/mL) in 50 mM Tris-HCl buffer (pH 8.6) as a substrate was added to each well, followed by incubation for 20 minutes. Next, the elastase activity inhibition rate in the B16-F10 melanocytes was measured. The absorbance of each of the sample-treated group and the sample-untreated group was measured, and the elastase activity inhibition rate was calculated using the following equation. The results are shown in Table 4 below.

$$Elastase activity inhibition rate ( \% ) = ( 1 - absorbance of sample treated group \text{} / absorbance of sample untreated group ) \times 100$$

TABLE 4
Results of Test Example 4

| | | Elastase activity |
|---|---|---|
| | | inhibition rate (%) |
| Example 1 | 0.2 mg/ml | 15.32 |
| | 1.0 mg/ml | 23.24 |
| | 5.0 mg/ml | 36.25 |
| Example 2 | 0.2 mg/ml | 16.98 |
| | 1.0 mg/ml | 25.39 |
| | 5.0 mg/ml | 38.98 |
| Example 3 | 0.2 mg/ml | 17.01 |
| | 1.0 mg/ml | 25.43 |
| | 5.0 mg/ml | 39.02 |
| Comparative | 0.2 mg/ml | 12.58 |
| Example 1 | 1.0 mg/ml | 20.51 |
| | 5.0 mg/ml | 30.25 |
| Vitamin C | 0.2 mg/ml | 36.78 |
| | 1.0 mg/ml | 75.25 |
| | 5.0 mg/ml | 90.55 |

As shown in Table 4 above, it could be confirmed that the samples of Examples 1, 2 and 3 had excellent elastase activity inhibition rates, indicating that they have an anti-wrinkle effect.

### Test Example 5

Human skin fibroblasts were cultured in Dulbeccos' minimum essential medium supplemented with 10% fetal bovine serum and 1% penicillin and streptomycin up to passage 5, and the cells were treated with the sample of each of Examples 1, 2 and 3. The change in the amount of collagen synthesis following treatment with each sample was tested in vitro to evaluate the collagen synthesis promoting effect of each sample in the human skin fibroblasts. In addition, untreated DMEM medium was prepared as a control and used for comparison.

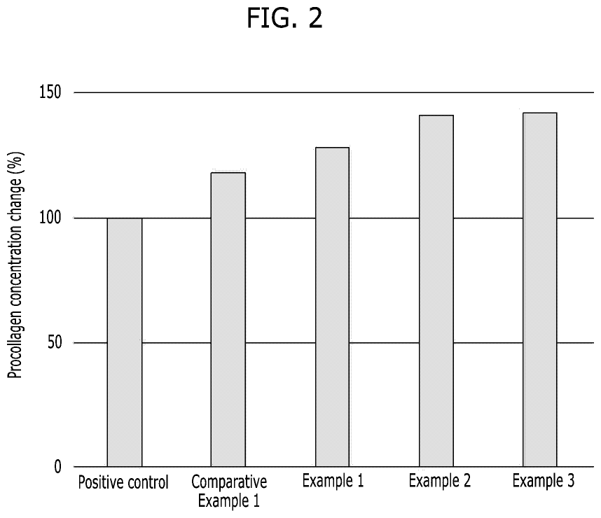

For each sample, human skin fibroblasts were seeded in a well plate at a density of 1×10⁴ cells/cm², and then incubated in an incubator (at 37° C. under 5% CO₂) for 24 hours for cell adhesion and environmental adaptation. After removal of the medium, the cells were treated with serum-free medium and starved for 8 hours. The sample of each Example was dissolved in a medium and the cells were treated with each sample solution. After sample treatment, the human skin fibroblasts were incubated for 72 hours, and the supernatant of the medium was collected and the synthesized collagen was quantified. Collagen quantification was performed using the Type I Procollagen C-Peptide EIA kit (Takara, Japan). The results are shown in FIG. 2.

As shown in FIG. 2, it could be confirmed that the samples of Examples 1, 2 and 3 of the present disclosure promoted collagen synthesis in human skin fibroblasts.

### Preparation Example 1

10 wt % of the sample of Example 1 and 90 wt % of maltodextrin were mixed together and formulated into tablets.

### Preparation Example 2

10 wt % of the sample of Example 2 and 90 wt % of maltodextrin were mixed together and formulated into tablets.

### Preparation Example 3

10 wt % of the sample of Example 3 and 90 wt % of maltodextrin were mixed together and formulated into tablets.

### Comparative Preparation Example 1

10 wt % of the sample of Comparative Example 1 and 90 wt % of maltodextrin were mixed together and formulated into tablets.

### Test Example 6

Ten men and women in their 10s to 60s for each sample were asked to consume 10 g of the sample of each of Preparation Examples 1, 2 and 3, Comparative Preparation Example 1, and a positive control after meals twice a day for 30 days. After consuming each sample for 30 days, the degree of reduction of fatigue feeling was evaluated on a five-point scale (5: very good, 4: good, 3: moderate, 2: poor, and 1: very poor) as shown in Table 5 below. Thereby, the effects of each sample on sound sleep, reduction in lethargy, and reduction in overall fatigue feeling were evaluated. As a positive control, commercially available vitamin C was provided. The results are shown in Table 5 below.

TABLE 5
Results of Test Example 6

| | Sound | Reduction | Reduction |
|---|---|---|---|
| | sleep | in | in fatigue |
| | effect | lethargy | feeling |
| Preparation Example 1 | 3.5 | 3.5 | 4.2 |
| Preparation Example 2 | 3.7 | 3.9 | 4.5 |
| Preparation Example 3 | 4.0 | 4.1 | 4.7 |
| Comparative | 3.0 | 3.6 | 3.5 |
| Preparation Example 1 | | | |
| Positive control | 2.0 | 2.1 | 2.2 |
| (vitamin C) | | | |

As shown in Table 5 above, it could be confirmed that the samples of Preparation Examples 1 to 3 of the present disclosure reduced fatigue feeling.

### Test Example 7

Fifteen women in their 50s for each sample were asked to consume 10 g of the sample of each of Preparation Examples 1, 2 and 3 after meals twice a day for 30 days. After consuming each sample for 30 days, the degrees of wrinkle reduction and whitening were examined. The results are shown in Table 6 below.

TABLE 6

| | | Not | Slightly | Moderately | Significantly |
|---|---|---|---|---|---|
| | | improved | improved | improved | improved |
| Wrinkle | Preparation | 0 | 4 | 8 | 3 |
| reduction | Example 1 | | | | |
| | Preparation | 0 | 2 | 10 | 3 |
| | Example 2 | | | | |
| | Preparation | 0 | 1 | 12 | 2 |
| | Example 3 | | | | |
| Whitening | Preparation | 0 | 9 | 6 | 0 |
| | Example 1 | | | | |
| | Preparation | 0 | 4 | 10 | 1 |
| | Example 2 | | | | |
| | Preparation | 0 | 4 | 9 | 2 |
| | Example 3 | | | | |

As shown in Table 6 above, it could be confirmed that the samples of Preparation Examples 1 to 3 were effective in wrinkle reduction and whitening. In addition, as a result of examining whether the samples had a fishy smell when consumed, the subjects all responded that they did not notice any unusual odor, indicating that the samples are also easy to consume.

The above description of the present disclosure is for illustrative purposes, and those of ordinary skill in the art will appreciate that the present disclosure can be easily modified into other specific forms without departing from the technical spirit or essential characteristics of the present invention. Therefore, the embodiments described above are considered to be illustrative in all respects and not restrictive.

What is claimed is:

1. A method for producing a plant-derived collagen peptide mixture, the method comprising steps of:
mixing Hibiscus and Tremella fuciformis;
adding water to the mixture, followed by heat treatment at 70 to 90° C. for 30 to 60 minutes;
adding an acidic solution to the heat-treated product, and adding protease thereto, followed by hydrolysis at 40 to 60° C. for 2 to 24 hours; and
deactivating the protease by heating the hydrolyzed solution at 75 to 85° C.

2. The method according to claim 1, wherein the step of mixing Hibiscus and Tremella fuciformis comprises mixing Hibiscus and Tremella fuciformis at a weight ratio of 1:0.5 to 1.

3. The method according to claim 1, further comprising, after the step of deactivating the protease, a step of mixing a fermentation extract of Pueraria flos and an extract of chicory with the deactivated solution.

4. The method according to claim 3, wherein the step of mixing the fermentation extract of Pueraria flos and the extract of chicory with the deactivated solution further comprises mixing an extract of kohlrabi.

5. The method according to claim 1, wherein the plant-derived collagen peptide mixture promotes collagen synthesis in vivo.
